(19) United States
(12) Patent Application Publication
(10) Pub. No.: US 2022/0341906 A1
(43) Pub. Date: Oct. 27, 2022

(54) Mobile Robot Environment Sensing

(71) Applicant: X Development LLC, Mountain View, CA (US)

(72) Inventors: Daniel Lam, Mountain View, CA (US); Justine Rembisz, San Carlos, CA (US); Asa Weiss, Standford, CA (US); Benjamin Holson, Sunnyvale, CA (US)

(21) Appl. No.: 17/240,232

(22) Filed: Apr. 26, 2021

(51) Int. Cl. G01N 33/00; G05D 1/02; G01K 3/14

(52) U.S. Cl. CPC G01N 33/0062; G01N 33/0047; G01N 33/004; G05D 1/0287; G05D 1/0212; G01N 33/0073; G01K 3/14; G05D 2201/0207

(57) ABSTRACT

A method includes receiving data collected by at least one sensor on a robotic device, wherein the data is to be used for an ambient environment state representation, and wherein the data represents ambient environment measurements collected at locations of the at least one sensor when the robotic device is passively monitoring an environment such that robotic device navigation is not based on the ambient environment state representation. The method further includes determining the ambient environment state representation using the data collected by the at least one sensor on the robotic device. The method also includes identifying, based on the ambient environment state representation, one or more anomalous ambient environment measurements. The method additionally includes causing, based on the one or more identified anomalous ambient environment measurements, the robotic device to actively monitor the environment such that robotic device navigation is based on the ambient environment state representation.

### BACKGROUND

As technology advances, various types of robotic devices are being created for performing a variety of functions that may assist users. Robotic devices may be used for applications involving material handling, transportation, welding, assembly, and dispensing, among others. Over time, the manner in which these robotic systems operate is becoming more intelligent, efficient, and intuitive. As robotic systems become increasingly prevalent in numerous aspects of modern life, it is desirable for robotic systems to be efficient. Therefore, a demand for efficient robotic systems has helped open up a field of innovation in actuators, movement, sensing techniques, as well as component design and assembly.

### SUMMARY

Example embodiments involve an environmental monitoring method for a robotic device. A robotic device may be equipped with at least one sensor. The robotic device may passively monitor environment properties while performing other tasks. When the robotic device detects an anomalous measurement, the robotic device may switch to actively monitor the environment such that robotic device navigation is based on the monitoring.

In an embodiment, a method includes receiving data collected by at least one sensor on a robotic device, wherein the data is to be used for an ambient environment state representation, and wherein the data represents ambient environment measurements collected at locations of the at least one sensor when the robotic device is passively monitoring an environment such that robotic device navigation is not based on the ambient environment state representation. The method further includes determining the ambient environment state representation using the data collected by the at least one sensor on the robotic device. The method also includes identifying, based on the ambient environment state representation, one or more anomalous ambient environment measurements. The method additionally includes causing, based on the one or more identified anomalous ambient environment measurements, the robotic device to actively monitor the environment such that robotic device navigation is based on the ambient environment state representation.

In another embodiment, a robotic device includes a control system and at least one sensor. The control system may be configured to receive data collected by the at least one sensor on a robotic device, wherein the data is to be used for an ambient environment state representation, and wherein the data represents ambient environment measurements collected at locations of the at least one sensor when the robotic device is passively monitoring an environment such that robotic device navigation is not based on the ambient environment state representation. The control system may be further configured to determine the ambient environment state representation using the data collected by the at least one sensor on the robotic device. The control system may also be configured to identify, based on the ambient environment state representation, one or more anomalous ambient environment measurements. The control system may additionally be configured to cause, based on the one or more identified anomalous ambient environment measurements, the robotic device to actively monitor the environment such that robotic device navigation is based on the ambient environment state representation.

In a further embodiment, a non-transitory computer readable medium is provided which includes programming instructions executable by at least one processor to cause the at least one processor to perform functions. The functions include receiving data collected by at least one sensor on a robotic device, wherein the data is to be used for an ambient environment state representation, and wherein the data represents ambient environment measurements collected at locations of the at least one sensor when the robotic device is passively monitoring an environment such that robotic device navigation is not based on the ambient environment state representation. The functions further include determining the ambient environment state representation using the data collected by the at least one sensor on the robotic device. The functions also include identifying, based on the ambient environment state representation, one or more anomalous ambient environment measurements. The functions additionally include causing, based on the one or more identified anomalous ambient environment measurements, the robotic device to actively monitor the environment such that robotic device navigation is based on the ambient environment state representation.

In a further embodiment, a system is provided that includes means for receiving data collected by at least one sensor on a robotic device, wherein the data is to be used for an ambient environment state representation, and wherein the data represents ambient environment measurements collected at locations of the at least one sensor when the robotic device is passively monitoring an environment such that robotic device navigation is not based on the ambient environment state representation. The system further includes means for determining the ambient environment state representation using the data collected by the at least one sensor on the robotic device. The system also includes means for identifying, based on the ambient environment state representation, one or more anomalous ambient environment measurements. The system additionally includes means for causing, based on the one or more identified anomalous ambient environment measurements, the robotic device to actively monitor the environment such that robotic device navigation is based on the ambient environment state representation.

The foregoing summary is illustrative only and is not intended to be in any way limiting. In addition to the illustrative aspects, embodiments, and features described above, further aspects, embodiments, and features will become apparent by reference to the figures and the following detailed description and the accompanying drawings.

### BRIEF DESCRIPTION OF THE DRAWINGS

FIG. 1 illustrates a configuration of a robotic system, in accordance with example embodiments.

FIG. 2 illustrates a mobile robot, in accordance with example embodiments.

FIG. 3 illustrates an exploded view of a mobile robot, in accordance with example embodiments.

FIG. 4 illustrates a robotic arm, in accordance with example embodiments.

FIG. 5 is a block diagram of a method, in accordance with example embodiments.

FIG. 6A illustrates an environment, in accordance with example embodiments.

FIG. 6B illustrates a table of measurements, in accordance with example embodiments.

FIG. 7A illustrates an ambient environment state representation, in accordance with example embodiments.

FIG. 7B illustrates an ambient environment state representation, in accordance with example embodiments.

FIG. 7C illustrates an ambient environment state representation, in accordance with example embodiments.

FIG. 7D illustrates an ambient environment state representation, in accordance with example embodiments.

### DETAILED DESCRIPTION

Example methods, devices, and systems are described herein. It should be understood that the words “example” and “exemplary” are used herein to mean “serving as an example, instance, or illustration.” Any embodiment or feature described herein as being an “example” or “exemplary” is not necessarily to be construed as preferred or advantageous over other embodiments or features unless indicated as such. Other embodiments can be utilized, and other changes can be made, without departing from the scope of the subject matter presented herein.

Thus, the example embodiments described herein are not meant to be limiting. It will be readily understood that the aspects of the present disclosure, as generally described herein, and illustrated in the figures, can be arranged, substituted, combined, separated, and designed in a wide variety of different configurations.

Throughout this description, the articles “a” or “an” are used to introduce elements of the example embodiments. Any reference to “a” or “an” refers to “at least one,” and any reference to “the” refers to “the at least one,” unless otherwise specified, or unless the context clearly dictates otherwise. The intent of using the conjunction “or” within a described list of at least two terms is to indicate any of the listed terms or any combination of the listed terms.

The use of ordinal numbers such as “first,” “second,” “third” and so on is to distinguish respective elements rather than to denote a particular order of those elements. For purpose of this description, the terms “multiple” and “a plurality of” refer to “two or more” or “more than one.”

Further, unless context suggests otherwise, the features illustrated in each of the figures may be used in combination with one another. Thus, the figures should be generally viewed as component aspects of one or more overall embodiments, with the understanding that not all illustrated features are necessary for each embodiment. In the figures, similar symbols typically identify similar components, unless context dictates otherwise. Further, unless otherwise noted, figures are not drawn to scale and are used for illustrative purposes only. Moreover, the figures are representational only and not all components are shown. For example, additional structural or restraining components might not be shown.

Additionally, any enumeration of elements, blocks, or steps in this specification or the claims is for purposes of clarity. Thus, such enumeration should not be interpreted to require or imply that these elements, blocks, or steps adhere to a particular arrangement or are carried out in a particular order.

### I. OVERVIEW

Traditionally, ambient properties of environments may be sporadically and/or sparsely monitored to provide application specific information. For example, temperature sensors may be placed sparsely around a building to monitor ambient temperature at static locations to determine thermostat adjustments. Humidity sensors may be sparsely placed in an environment to monitor the concentration of water vapor in the air, particularly in situations where water vapor may damage objects located in the environment. Air quality sensors, such as non-volatile and/or volatile organic compound sensors, may be placed temporarily at select locations to notify occupants of potentially harmful air conditions.

However, due to their sparse placements and static locations, these sensors may provide limited actionable information. Taking air quality monitoring as an example, air quality sensors may generally be expensive and unlikely to be integrated into existing infrastructure. Air quality monitoring and reporting may thus be performed manually every few months to circumvent these limitations. However, air quality could fluctuate drastically between these reports due to intermittent events (e.g., changes in cleaning supplies, forest fires, etc.). More frequent reporting and collecting data at more locations could thus provide additional relevant information, as well as more actionable reports.

Provided herein are methods to dynamically monitor ambient environment properties using robotic devices. In recent years, robotic devices are being developed to perform various repetitive and potentially labor intensive tasks, such as moving objects, vacuuming floors, etc. In some examples, sensors may be attached to or integrated into these robotic devices, and these sensors may be used to passively collect environmental data while the robotic device is performing these tasks. The passively collected data may be compiled into an ambient environment state representation. Based on the ambient environment state representation, the robot and/or a user may make decisions intended to mitigate or improve environmental conditions (e.g., changing cleaning supplies, adding an air purifier, etc.). This passive monitoring of the environment may facilitate obtaining a more complete understanding of the ambient environment.

In some examples, the robotic device may use the ambient environment state representation as a basis to identify whether one or more measurements are anomalous. In response to identifying one or more anomalous ambient environment measurements, the robotic device may switch to actively monitoring the environment, which may involve adjusting robot navigation to update the ambient state representation and/or to detect a source of the one or more anomalous ambient environment measurements.

Various sensors may be used to collect the ambient environment measurements, including but not limited to, temperature, humidity, total volatile organic compounds, particulate matter, carbon dioxide, and WiFi. These sensors may be integrated into the robotic device in a permanent manner (e.g., soldered on a printed circuit board or fixed in place during a manufacturing process). Alternatively, these sensors may be integrated onto the robotic device in a non-permanent, removable, and perhaps interchangeable manner (e.g., the robotic device may have integrated female headers and sensors may have corresponding male headers). A robotic device may be able to connect and disconnect these removable sensors based on collected sensor data. For example, a robotic device may change sensors to detect other properties of the environment (e.g., disconnecting a temperature sensor, then connecting an air quality sensor) or to replace sensors that were determined to be inaccurate.

While such a robotic device is performing another task, e.g., sanitizing surfaces, the robotic device may use the integrated sensors to passively monitor the environment and collect ambient environment data. The ambient environment data may consist of ambient environment measurements, which may be compiled into an ambient environment state representation. In some examples, a robotic device may receive the ambient environment measurements at different locations and at different points in time. The robotic device may receive coordinate locations indicative of where the ambient environment measurements were collected and timestamps indicative of when the ambient environment measurements were collected. The ambient environment measurements, locations, and timestamps may then be compiled into an ambient environment representation.

In some examples, the ambient environment representation may incorporate a floorplan of the environment. The floorplan of the environment could be stored in memory on the robotic device or derived from a map that the robot uses for navigation. The measurements collected, including the ambient environment measurements, collection locations, and/or timestamps, may be depicted in a visual representation, e.g., a heat map, perhaps incorporating the floorplan. For example, ambient environment measurements from a specific sensor may be analyzed and assigned a specific color relative to a threshold or relative to the other ambient environment measurements. Using the collected coordinate locations, the ambient environment measurements may be located on the floorplan, and each ambient environment measurement may be depicted as a point or other symbol with the assigned color on the floorplan. Further, an ambient environment representation may only incorporate points of a certain timeframe, and measurements collected outside the certain timeframe may be incorporated into a separate ambient environment representation. Alternatively, the ambient environment representation may incorporate several different timeframes. Further, the ambient environment representation may incorporate ambient environment measurements from a single sensor or multiple sensors on the robotic device.

Additionally or alternatively, the ambient environment representation may incorporate a table or database indicating the ambient environment measurements. The table may additionally include locations, perhaps in the form of coordinates including X, Y, and Z data or in the form of room numbers/names. The table may further include timestamps of the year, the date, and/or the time at which the data was collected. For example, the table may record that a temperature of 70 degrees Fahrenheit was reported in Room 52, on Mar. 4, 2021.

Based on the ambient environment representation, one or more anomalous measurements may be identified. Recently reported ambient environment measurements may be compared with past measurements using the ambient environment representation to determine whether the recently reported measurements are anomalous with respect to sensor type, time, and/or location. For example, based on the measurements depicted by the ambient environment representation, the robotic device may determine that temperatures in the environment are typically between 65 degrees to 75 degrees Fahrenheit, and the robotic device may determine that subsequent measurements outside of that range are anomalous. In further examples, the robotic device may determine that for certain areas of the map, temperatures have not exceeded 50 degrees Fahrenheit for more than ten measurements, and therefore, temperatures above 50 degrees Fahrenheit in that particular area may be considered anomalous. Other thresholds are possible. Further, recently reported ambient environment measurements may be compared with past measurements depicted in the ambient environment representation through the use of statistical tests, for example, t-tests.

In response to identifying one or more anomalous measurements, the robotic device may respond in a variety of ways. In some examples, the robotic device may send the anomalous measurements to a remote device along with other relevant information, e.g., where the anomalous measurements were collected, at what time the anomalous measurements were collected, and the type of measurement. Additionally or alternatively, the robotic device may send a notification to a user device, notifying the user that one or more anomalous measurements were detected.

In some examples, in response to identifying one or more anomalous measurements, the robotic device may switch to active monitoring. As mentioned above, before the robotic device detected one or more anomalous measurements, the robotic device may have been performing various tasks while passively monitoring the environment and collecting ambient environment data. Upon detection of one or more anomalous measurements, however, the robotic device may switch to active monitoring such that the robotic device may actively seek to update the ambient environment representation and/or actively seek out the source of the anomalous measurements.

In some examples, in response to identifying that one or more anomalous measurements have been detected, the robotic device may adjust robot navigation to collect additional sensor data in order to update the ambient environment representation. For example, the robotic device may recognize location gaps between measurements in the ambient environment representation, and the robotic device may actively navigate to resolve these gaps in the ambient environment representation.

In further examples, upon detecting that one or more anomalous measurements have been detected, the robotic device may actively attempt to detect the source of the anomalous measurements. The robotic device may thus determine, perhaps using the ambient environment state representation, a possible direction in which the source of the anomalous measurement may be located. The robotic device may navigate towards this direction, collect another measurement, and determine whether the collected measurement is increasingly anomalous or decreasingly anomalous. If the collected measurement is increasingly anomalous, the robotic device may move further in the determined direction. If the collected measurement is decreasingly anomalous, the robotic device may move in the opposite direction or in another direction. The movement of the robotic device may be refined in this way until the source of the one or more anomalous measurements is approximately or fully located. In some situations, the robotic device may move an appendage on which a sensor is located to further refine the location. After the source of the one or more anomalous measurements is located, the robotic device may transmit the location of the source of the one or more anomalous measurements to a remote device.

In some examples, after determining a location of the source of the anomalous measurements, the robotic device may capture additional sensor data with an additional sensor, which may be cross-referenced with the anomalous measurements to identify the source of the anomalous measurements. For example, the robotic device may have detected anomalous air quality measurements and located a local maximum where the air quality measurements are most anomalous. Subsequently, the robotic device may use a camera to identify the source of the anomalous air quality measurements. For example, the robotic device may capture an image including a puddle of water and a burning cigarette. By cross-referencing the anomalous measurements, the robotic device may determine that the source of poor air quality is the burning cigarette. The robotic device may then transmit the camera images, perhaps only those identifying the source of the captured image, to a remote device. The robotic device may additionally transmit the determined identity of the source, e.g., “cigarette.”

In some examples, after detecting the source of the anomalous measurements, the robotic device may use further sensor data captured by another sensor to find the downstream effects of the source of the anomalous ambient environment measurements. For example, the robotic device may be monitoring air quality. The robotic device may have detected one or more anomalous air quality measurements, and the robotic device may have located a local maximum where the air quality measurements are most anomalous. Using another sensor, e.g., an air flow sensor, the robotic device may detect a direction in which air (and particulate matter in the air) is flowing. Based on the determined direction of air flow, the robotic device may also collect additional air quality measurements indicative of the downstream effects of the source of anomalous measurements. The additional air quality measurements may be used to update the ambient environment state representation, which may be sent to a remote device after the updates are complete.

### II. EXAMPLE ROBOTIC SYSTEMS

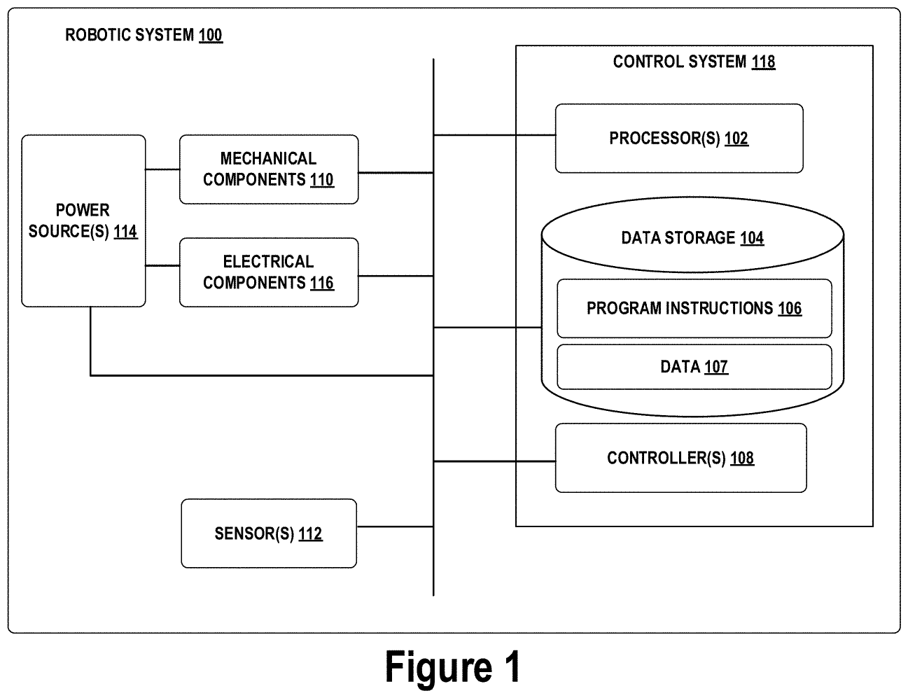

FIG. 1 illustrates an example configuration of a robotic system that may be used in connection with the implementations described herein. Robotic system 100 may be configured to operate autonomously, semi-autonomously, or using directions provided by user(s). Robotic system 100 may be implemented in various forms, such as a robotic arm, industrial robot, or some other arrangement. Some example implementations involve a robotic system 100 engineered to be low cost at scale and designed to support a variety of tasks. Robotic system 100 may be designed to be capable of operating around people. Robotic system 100 may also be optimized for machine learning. Throughout this description, robotic system 100 may also be referred to as a robot, robotic device, or mobile robot, among other designations.

As shown in FIG. 1, robotic system 100 may include processor(s) 102, data storage 104, and controller(s) 108, which together may be part of control system 118. Robotic system 100 may also include sensor(s) 112, power source(s) 114, mechanical components 110, and electrical components 116. Nonetheless, robotic system 100 is shown for illustrative purposes, and may include more or fewer components. The various components of robotic system 100 may be connected in any manner, including wired or wireless connections. Further, in some examples, components of robotic system 100 may be distributed among multiple physical entities rather than a single physical entity. Other example illustrations of robotic system 100 may exist as well.

Processor(s) 102 may operate as one or more general-purpose hardware processors or special purpose hardware processors (e.g., digital signal processors, application specific integrated circuits, etc.). Processor(s) 102 may be configured to execute computer-readable program instructions 106, and manipulate data 107, both of which are stored in data storage 104. Processor(s) 102 may also directly or indirectly interact with other components of robotic system 100, such as sensor(s) 112, power source(s) 114, mechanical components 110, or electrical components 116.

Data storage 104 may be one or more types of hardware memory. For example, data storage 104 may include or take the form of one or more computer-readable storage media that can be read or accessed by processor(s) 102. The one or more computer-readable storage media can include volatile or non-volatile storage components, such as optical, magnetic, organic, or another type of memory or storage, which can be integrated in whole or in part with processor(s) 102. In some implementations, data storage 104 can be a single physical device. In other implementations, data storage 104 can be implemented using two or more physical devices, which may communicate with one another via wired or wireless communication. As noted previously, data storage 104 may include the computer-readable program instructions 106 and data 107. Data 107 may be any type of data, such as configuration data, sensor data, or diagnostic data, among other possibilities.

Controller 108 may include one or more electrical circuits, units of digital logic, computer chips, or microprocessors that are configured to (perhaps among other tasks), interface between any combination of mechanical components 110, sensor(s) 112, power source(s) 114, electrical components 116, control system 118, or a user of robotic system 100. In some implementations, controller 108 may be a purpose-built embedded device for performing specific operations with one or more subsystems of the robotic system 100.

Control system 118 may monitor and physically change the operating conditions of robotic system 100. In doing so, control system 118 may serve as a link between portions of robotic system 100, such as between mechanical components 110 or electrical components 116. In some instances, control system 118 may serve as an interface between robotic system 100 and another computing device. Further, control system 118 may serve as an interface between robotic system 100 and a user. In some instances, control system 118 may include various components for communicating with robotic system 100, including a joystick, buttons, or ports, etc. The example interfaces and communications noted above may be implemented via a wired or wireless connection, or both. Control system 118 may perform other operations for robotic system 100 as well.

During operation, control system 118 may communicate with other systems of robotic system 100 via wired or wireless connections, and may further be configured to communicate with one or more users of the robot. As one possible illustration, control system 118 may receive an input (e.g., from a user or from another robot) indicating an instruction to perform a requested task, such as to pick up and move an object from one location to another location. Based on this input, control system 118 may perform operations to cause the robotic system 100 to make a sequence of movements to perform the requested task. As another illustration, a control system may receive an input indicating an instruction to move to a requested location. In response, control system 118 (perhaps with the assistance of other components or systems) may determine a direction and speed to move robotic system 100 through an environment en route to the requested location.

Operations of control system 118 may be carried out by processor(s) 102. Alternatively, these operations may be carried out by controller(s) 108, or a combination of processor(s) 102 and controller(s) 108. In some implementations, control system 118 may partially or wholly reside on a device other than robotic system 100, and therefore may at least in part control robotic system 100 remotely.

Mechanical components 110 represent hardware of robotic system 100 that may enable robotic system 100 to perform physical operations. As a few examples, robotic system 100 may include one or more physical members, such as an arm, an end effector, a head, a neck, a torso, a base, and wheels. The physical members or other parts of robotic system 100 may further include actuators arranged to move the physical members in relation to one another. Robotic system 100 may also include one or more structured bodies for housing control system 118 or other components, and may further include other types of mechanical components. The particular mechanical components 110 used in a given robot may vary based on the design of the robot, and may also be based on the operations or tasks the robot may be configured to perform.

In some examples, mechanical components 110 may include one or more removable components. Robotic system 100 may be configured to add or remove such removable components, which may involve assistance from a user or another robot. For example, robotic system 100 may be configured with removable end effectors or digits that can be replaced or changed as needed or desired. In some implementations, robotic system 100 may include one or more removable or replaceable battery units, control systems, power systems, bumpers, or sensors. Other types of removable components may be included within some implementations.

Robotic system 100 may include sensor(s) 112 arranged to sense aspects of robotic system 100. Sensor(s) 112 may include one or more force sensors, torque sensors, velocity sensors, acceleration sensors, position sensors, proximity sensors, motion sensors, location sensors, load sensors, temperature sensors, touch sensors, depth sensors, ultrasonic range sensors, infrared sensors, object sensors, or cameras, among other possibilities. Within some examples, robotic system 100 may be configured to receive sensor data from sensors that are physically separated from the robot (e.g., sensors that are positioned on other robots or located within the environment in which the robot is operating).

Sensor(s) 112 may provide sensor data to processor(s) 102 (perhaps by way of data 107) to allow for interaction of robotic system 100 with its environment, as well as monitoring of the operation of robotic system 100. The sensor data may be used in evaluation of various factors for activation, movement, and deactivation of mechanical components 110 and electrical components 116 by control system 118. For example, sensor(s) 112 may capture data corresponding to the terrain of the environment or location of nearby objects, which may assist with environment recognition and navigation.

In some examples, sensor(s) 112 may include RADAR (e.g., for long-range object detection, distance determination, or speed determination), LIDAR (e.g., for short-range object detection, distance determination, or speed determination), SONAR (e.g., for underwater object detection, distance determination, or speed determination), VICON® (e.g., for motion capture), one or more cameras (e.g., stereoscopic cameras for 3D vision), a global positioning system (GPS) transceiver, or other sensors for capturing information of the environment in which robotic system 100 is operating. Sensor(s) 112 may monitor the environment in real time, and detect obstacles, elements of the terrain, weather conditions, temperature, or other aspects of the environment. In another example, sensor(s) 112 may capture data corresponding to one or more characteristics of a target or identified object, such as a size, shape, profile, structure, or orientation of the object.

Further, robotic system 100 may include sensor(s) 112 configured to receive information indicative of the state of robotic system 100, including sensor(s) 112 that may monitor the state of the various components of robotic system 100. Sensor(s) 112 may measure activity of systems of robotic system 100 and receive information based on the operation of the various features of robotic system 100, such as the operation of an extendable arm, an end effector, or other mechanical or electrical features of robotic system 100. The data provided by sensor(s) 112 may enable control system 118 to determine errors in operation as well as monitor overall operation of components of robotic system 100.

As an example, robotic system 100 may use force/torque sensors to measure load on various components of robotic system 100. In some implementations, robotic system 100 may include one or more force/torque sensors on an arm or end effector to measure the load on the actuators that move one or more members of the arm or end effector. In some examples, the robotic system 100 may include a force/torque sensor at or near the wrist or end effector, but not at or near other joints of a robotic arm. In further examples, robotic system 100 may use one or more position sensors to sense the position of the actuators of the robotic system. For instance, such position sensors may sense states of extension, retraction, positioning, or rotation of the actuators on an arm or end effector.

As another example, sensor(s) 112 may include one or more velocity or acceleration sensors. For instance, sensor(s) 112 may include an inertial measurement unit (IMU). The IMU may sense velocity and acceleration in the world frame, with respect to the gravity vector. The velocity and acceleration sensed by the IMU may then be translated to that of robotic system 100 based on the location of the IMU in robotic system 100 and the kinematics of robotic system 100.

Robotic system 100 may include other types of sensors not explicitly discussed herein. Additionally or alternatively, the robotic system may use particular sensors for purposes not enumerated herein.

Robotic system 100 may also include one or more power source(s) 114 configured to supply power to various components of robotic system 100. Among other possible power systems, robotic system 100 may include a hydraulic system, electrical system, batteries, or other types of power systems. As an example illustration, robotic system 100 may include one or more batteries configured to provide charge to components of robotic system 100. Some of mechanical components 110 or electrical components 116 may each connect to a different power source, may be powered by the same power source, or be powered by multiple power sources.

Any type of power source may be used to power robotic system 100, such as electrical power or a gasoline engine. Additionally or alternatively, robotic system 100 may include a hydraulic system configured to provide power to mechanical components 110 using fluid power. Components of robotic system 100 may operate based on hydraulic fluid being transmitted throughout the hydraulic system to various hydraulic motors and hydraulic cylinders, for example. The hydraulic system may transfer hydraulic power by way of pressurized hydraulic fluid through tubes, flexible hoses, or other links between components of robotic system 100. Power source(s) 114 may charge using various types of charging, such as wired connections to an outside power source, wireless charging, combustion, or other examples.

Electrical components 116 may include various mechanisms capable of processing, transferring, or providing electrical charge or electric signals. Among possible examples, electrical components 116 may include electrical wires, circuitry, or wireless communication transmitters and receivers to enable operations of robotic system 100. Electrical components 116 may interwork with mechanical components 110 to enable robotic system 100 to perform various operations. Electrical components 116 may be configured to provide power from power source(s) 114 to the various mechanical components 110, for example. Further, robotic system 100 may include electric motors. Other examples of electrical components 116 may exist as well.

Robotic system 100 may include a body, which may connect to or house appendages and components of the robotic system. As such, the structure of the body may vary within examples and may further depend on particular operations that a given robot may have been designed to perform. For example, a robot developed to carry heavy loads may have a wide body that enables placement of the load. Similarly, a robot designed to operate in tight spaces may have a relatively tall, narrow body. Further, the body or the other components may be developed using various types of materials, such as metals or plastics. Within other examples, a robot may have a body with a different structure or made of various types of materials.

The body or the other components may include or carry sensor(s) 112. These sensors may be positioned in various locations on the robotic system 100, such as on a body, a head, a neck, a base, a torso, an arm, or an end effector, among other examples.

Robotic system 100 may be configured to carry a load, such as a type of cargo that is to be transported. In some examples, the load may be placed by the robotic system 100 into a bin or other container attached to the robotic system 100. The load may also represent external batteries or other types of power sources (e.g., solar panels) that the robotic system 100 may utilize. Carrying the load represents one example use for which the robotic system 100 may be configured, but the robotic system 100 may be configured to perform other operations as well.

As noted above, robotic system 100 may include various types of appendages, wheels, end effectors, gripping devices and so on. In some examples, robotic system 100 may include a mobile base with wheels, treads, or some other form of locomotion. Additionally, robotic system 100 may include a robotic arm or some other form of robotic manipulator. In the case of a mobile base, the base may be considered as one of mechanical components 110 and may include wheels, powered by one or more of actuators, which allow for mobility of a robotic arm in addition to the rest of the body.

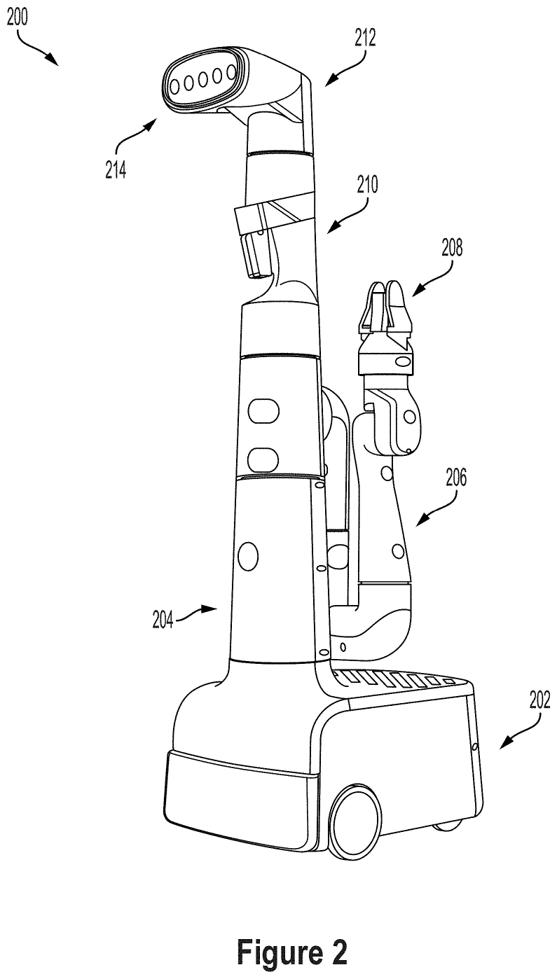

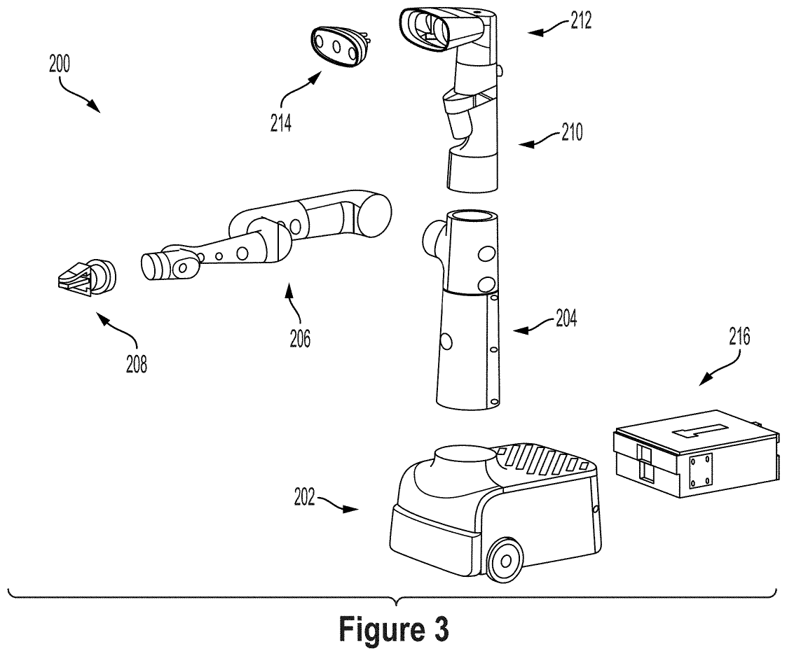

FIG. 2 illustrates a mobile robot, in accordance with example embodiments. FIG. 3 illustrates an exploded view of the mobile robot, in accordance with example embodiments. More specifically, a robot 200 may include a mobile base 202, a midsection 204, an arm 206, an end-of-arm system (EOAS) 208, a mast 210, a perception housing 212, and a perception suite 214. The robot 200 may also include a compute box 216 stored within mobile base 202.

The mobile base 202 includes two drive wheels positioned at a front end of the robot 200 in order to provide locomotion to robot 200. The mobile base 202 also includes additional casters (not shown) to facilitate motion of the mobile base 202 over a ground surface. The mobile base 202 may have a modular architecture that allows compute box 216 to be easily removed. Compute box 216 may serve as a removable control system for robot 200 (rather than a mechanically integrated control system). After removing external shells, the compute box 216 can be easily removed and/or replaced. The mobile base 202 may also be designed to allow for additional modularity. For example, the mobile base 202 may also be designed so that a power system, a battery, and/or external bumpers can all be easily removed and/or replaced.

The midsection 204 may be attached to the mobile base 202 at a front end of the mobile base 202. The midsection 204 includes a mounting column which is fixed to the mobile base 202. The midsection 204 additionally includes a rotational joint for arm 206. More specifically, the midsection 204 includes the first two degrees of freedom for arm 206 (a shoulder yaw J0 joint and a shoulder pitch J1 joint). The mounting column and the shoulder yaw J0 joint may form a portion of a stacked tower at the front of mobile base 202. The mounting column and the shoulder yaw J0 joint may be coaxial. The length of the mounting column of midsection 204 may be chosen to provide the arm 206 with sufficient height to perform manipulation tasks at commonly encountered height levels (e.g., coffee table top and counter top levels). The length of the mounting column of midsection 204 may also allow the shoulder pitch J1 joint to rotate the arm 206 over the mobile base 202 without contacting the mobile base 202.

The arm 206 may be a 7DOF robotic arm when connected to the midsection 204. As noted, the first two DOFs of the arm 206 may be included in the midsection 204. The remaining five DOFs may be included in a standalone section of the arm 206 as illustrated in FIGS. 2 and 3. The arm 206 may be made up of plastic monolithic link structures. Inside the arm 206 may be housed standalone actuator modules, local motor drivers, and thru bore cabling.

The EOAS 208 may be an end effector at the end of arm 206. EOAS 208 may allow the robot 200 to manipulate objects in the environment. As shown in FIGS. 2 and 3, EOAS 208 may be a gripper, such as an underactuated pinch gripper. The gripper may include one or more contact sensors such as force/torque sensors and/or non-contact sensors such as one or more cameras to facilitate object detection and gripper control. EOAS 208 may also be a different type of gripper such as a suction gripper or a different type of tool such as a drill or a brush. EOAS 208 may also be swappable or include swappable components such as gripper digits.

The mast 210 may be a relatively long, narrow component between the shoulder yaw J0 joint for arm 206 and perception housing 212. The mast 210 may be part of the stacked tower at the front of mobile base 202. The mast 210 may be fixed relative to the mobile base 202. The mast 210 may be coaxial with the midsection 204. The length of the mast 210 may facilitate perception by perception suite 214 of objects being manipulated by EOAS 208. The mast 210 may have a length such that when the shoulder pitch J1 joint is rotated vertical up, a topmost point of a bicep of the arm 206 is approximately aligned with a top of the mast 210. The length of the mast 210 may then be sufficient to prevent a collision between the perception housing 212 and the arm 206 when the shoulder pitch J1 joint is rotated vertical up.

As shown in FIGS. 2 and 3, the mast 210 may include a 3D lidar sensor configured to collect depth information about the environment. The 3D lidar sensor may be coupled to a carved-out portion of the mast 210 and fixed at a downward angle. The lidar position may be optimized for localization, navigation, and for front cliff detection.

The perception housing 212 may include at least one sensor making up perception suite 214. The perception housing 212 may be connected to a pan/tilt control to allow for reorienting of the perception housing 212 (e.g., to view objects being manipulated by EOAS 208). The perception housing 212 may be a part of the stacked tower fixed to the mobile base 202. A rear portion of the perception housing 212 may be coaxial with the mast 210.

The perception suite 214 may include a suite of sensors configured to collect sensor data representative of the environment of the robot 200. The perception suite 214 may include an infrared(IR)-assisted stereo depth sensor. The perception suite 214 may additionally include a wide-angled red-green-blue (RGB) camera for human-robot interaction and context information. The perception suite 214 may additionally include a high resolution RGB camera for object classification. A face light ring surrounding the perception suite 214 may also be included for improved human-robot interaction and scene illumination. In some examples, the perception suite 214 may also include a projector configured to project images and/or video into the environment.

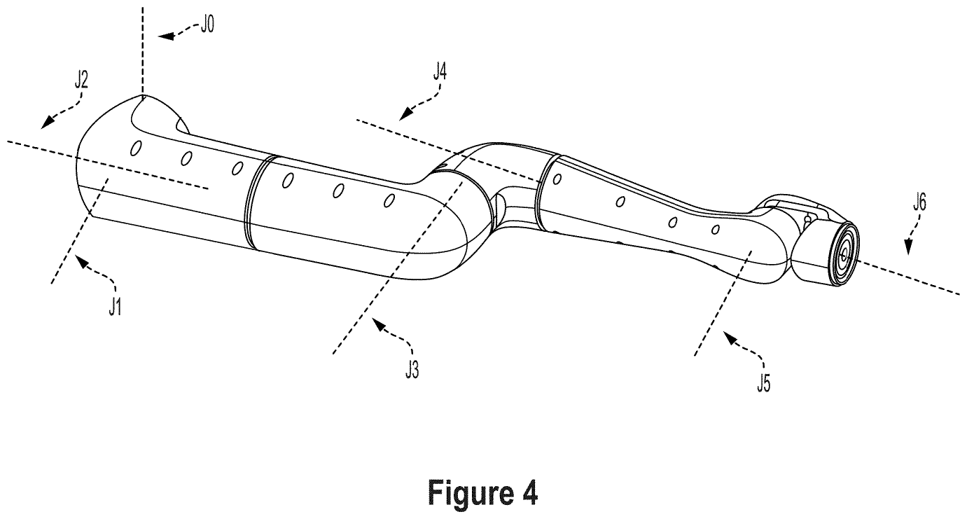

FIG. 4 illustrates a robotic arm, in accordance with example embodiments. The robotic arm includes 7 DOFs: a shoulder yaw J0 joint, a shoulder pitch J1 joint, a bicep roll J2 joint, an elbow pitch J3 joint, a forearm roll J4 joint, a wrist pitch J5 joint, and wrist roll J6 joint. Each of the joints may be coupled to one or more actuators. The actuators coupled to the joints may be operable to cause movement of links down the kinematic chain (as well as any end effector attached to the robot arm).

The shoulder yaw J0 joint allows the robot arm to rotate toward the front and toward the back of the robot. One beneficial use of this motion is to allow the robot to pick up an object in front of the robot and quickly place the object on the rear section of the robot (as well as the reverse motion). Another beneficial use of this motion is to quickly move the robot arm from a stowed configuration behind the robot to an active position in front of the robot (as well as the reverse motion).

The shoulder pitch J1 joint allows the robot to lift the robot arm (e.g., so that the bicep is up to perception suite level on the robot) and to lower the robot arm (e.g., so that the bicep is just above the mobile base). This motion is beneficial to allow the robot to efficiently perform manipulation operations (e.g., top grasps and side grasps) at different target height levels in the environment. For instance, the shoulder pitch J1 joint may be rotated to a vertical up position to allow the robot to easily manipulate objects on a table in the environment. The shoulder pitch J1 joint may be rotated to a vertical down position to allow the robot to easily manipulate objects on a ground surface in the environment.

The bicep roll J2 joint allows the robot to rotate the bicep to move the elbow and forearm relative to the bicep. This motion may be particularly beneficial for facilitating a clear view of the EOAS by the robot's perception suite. By rotating the bicep roll J2 joint, the robot may kick out the elbow and forearm to improve line of sight to an object held in a gripper of the robot.

Moving down the kinematic chain, alternating pitch and roll joints (a shoulder pitch J1 joint, a bicep roll J2 joint, an elbow pitch J3 joint, a forearm roll J4 joint, a wrist pitch J5 joint, and wrist roll J6 joint) are provided to improve the manipulability of the robotic arm. The axes of the wrist pitch J5 joint, the wrist roll J6 joint, and the forearm roll J4 joint are intersecting for reduced arm motion to reorient objects. The wrist roll J6 point is provided instead of two pitch joints in the wrist in order to improve object rotation.

In some examples, a robotic arm such as the one illustrated in FIG. 4 may be capable of operating in a teach mode. In particular, teach mode may be an operating mode of the robotic arm that allows a user to physically interact with and guide robotic arm towards carrying out and recording various movements. In a teaching mode, an external force is applied (e.g., by the user) to the robotic arm based on a teaching input that is intended to teach the robot regarding how to carry out a specific task. The robotic arm may thus obtain data regarding how to carry out the specific task based on instructions and guidance from the user. Such data may relate to a plurality of configurations of mechanical components, joint position data, velocity data, acceleration data, torque data, force data, and power data, among other possibilities.

During teach mode the user may grasp onto the EOAS or wrist in some examples or onto any part of robotic arm in other examples, and provide an external force by physically moving robotic arm. In particular, the user may guide the robotic arm towards grasping onto an object and then moving the object from a first location to a second location. As the user guides the robotic arm during teach mode, the robot may obtain and record data related to the movement such that the robotic arm may be configured to independently carry out the task at a future time during independent operation (e.g., when the robotic arm operates independently outside of teach mode). In some examples, external forces may also be applied by other entities in the physical workspace such as by other objects, machines, or robotic systems, among other possibilities.

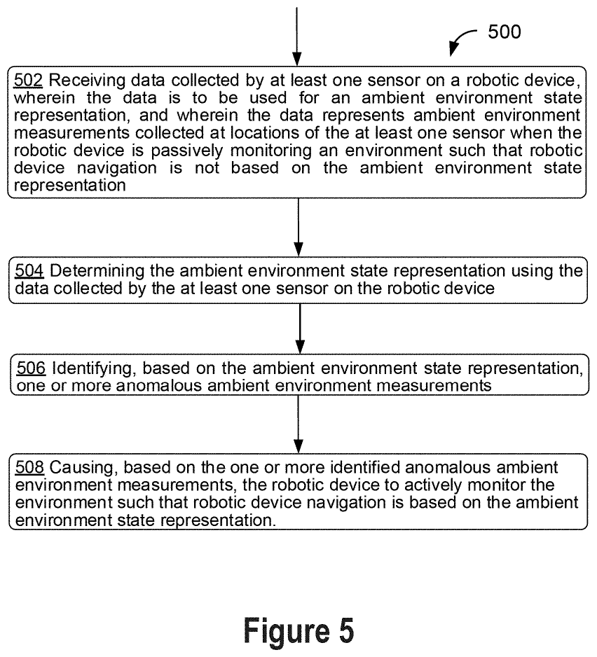

FIG. 5 is a block diagram of method 500, in accordance with example embodiments. Blocks 502, 504, 506, and 508 may collectively be referred to as method 500. In some examples, method 500 of FIG. 5 may be carried out by a control system, such as control system 118 of robotic system 100. In further examples, method 500 of FIG. 5 may be carried out by a computing device or a server device remote from the robotic device. In still further examples, method 500 may be carried out by one or more processors, such as processor(s) 102, executing program instructions, such as program instructions 106, stored in a data storage, such as data storage 104. Execution of method 500 may involve a robotic device, such as the robotic device illustrated and described with respect to FIGS. 1-4. Further, execution of method 500 may involve a computing device or a server device remote from the robotic device and robotic system 100. Other robotic devices may also be used in the performance of method 500. In further examples, some or all of the blocks of method 500 may be performed by a control system remote from the robotic device. In yet further examples, different blocks of method 500 may be performed by different control systems, located on and/or remote from a robotic device.

Those skilled in the art will understand that the block diagram of FIG. 5 illustrates functionality and operation of certain implementations of the present disclosure. In this regard, each block of the block diagram may represent a module, a segment, or a portion of program code, which includes one or more instructions executable by one or more processors for implementing specific logical functions or steps in the process. The program code may be stored on any type of computer readable medium, for example, such as a storage device including a disk or hard drive.

In addition, each block may represent circuitry that is wired to perform the specific logical functions in the process. Alternative implementations are included within the scope of the example implementations of the present application in which functions may be executed out of order from that shown or discussed, including substantially concurrent or in reverse order, depending on the functionality involved, as would be understood by those reasonably skilled in the art.

At block 502, method 500 includes receiving data collected by at least one sensor on a robotic device. The data is to be used for an ambient environment state representation. The data represents ambient environment measurements collected at locations of the at least one sensor when the robotic device is passively monitoring an environment such that robotic device navigation is not based on the ambient environment state representation.

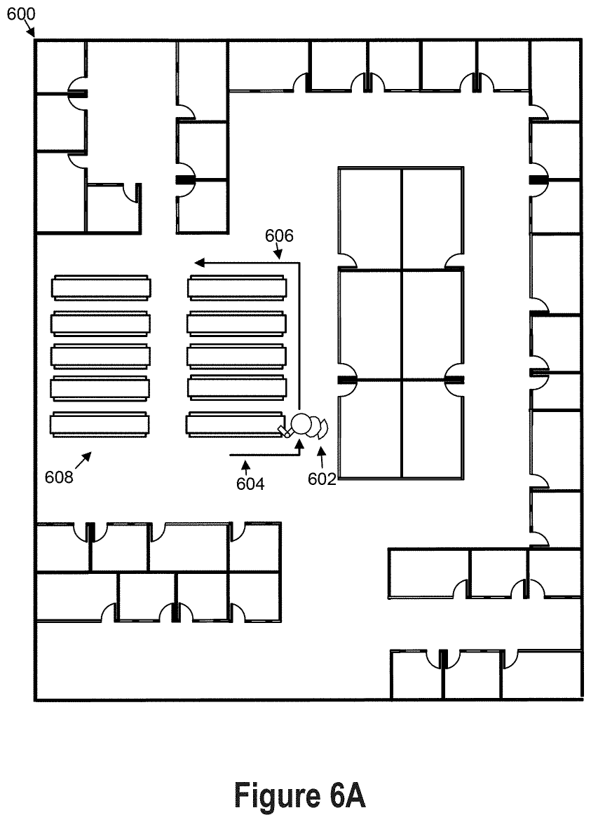

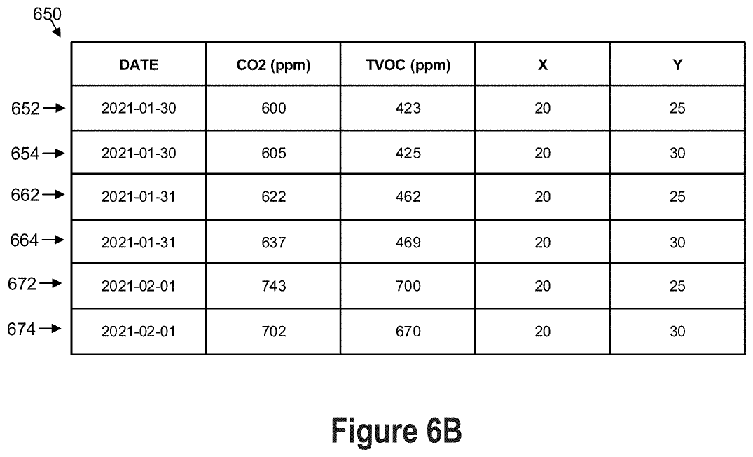

FIG. 6A depicts a robotic device 602 passively monitoring environment 600, in accordance with example embodiments. At least one sensor may be integrated into robotic device 602 and the sensor may be configured to monitor an ambient environment property. Example sensors may include those with the ability to monitor temperature, humidity, air quality, air flow, compounds in the air (e.g., carbon dioxide, volatile organic compounds, non-volatile organic compounds), particulate matter, or wireless network strength.

The sensor may be located in a variety of locations on the robotic device. In some examples, robotic device 602 may have a similar architecture to robot 200. The sensor may be integrated into and/or proximate to mobile base 202, midsection 204, arm 206, EOAS 208, mast 210, perception housing 212, and/or perception suite 214. As mentioned above, the sensors may be integrated in a permanent manner (e.g., soldered onto a printed circuit board or otherwise fixed during the manufacturing process) or a permanent manner a non-permanent, removable, and perhaps interchangeable manner (e.g., the robotic device may have integrated female headers and sensors may have corresponding male headers, or vice versa).

In some examples, robotic device 602 may be operating in environment 600 and may be performing tasks within environment 600, e.g., clearing tables 608. In clearing tables 608, robotic device 602 may passively collect measurements representative of one or more ambient environment properties such that the navigation of robotic device 602 is not dependent on the measurements collected. For example, robotic device 602 may be clearing tables while moving along path 604 and may collect a carbon dioxide measurement before, during, and/or after clearing a table. After clearing the table and after collecting the carbon dioxide measurement, the robotic device may navigate to clear another table along path 606. This navigation may be predetermined, based on a map that the robotic device has access to, perhaps indicating the tables that the robotic device has not yet cleared, and/or based on sensor measurements from other sensors, perhaps illustrating that a table has already been cleaned. Additionally or alternatively, the robotic device may be performing other tasks in environment 600, e.g., cleaning tables, emptying trash, vacuuming, and/or greeting people.

The collected ambient environment measurements may be aggregated into a database or other table stored in memory of robotic device 602 or in a remote server. FIG. 6B depicts table 650 of collected measurements, in accordance with example embodiments. Table 650 includes carbon dioxide (CO2) measurements and total volatile organic compounds (TVOC) measurements that were passively collected while the robotic device was performing another task. Additionally, table 650 includes location measurements in the form of coordinates (as indicated by X-axis and Y-axis measurements) as well as date stamps indicating the date in which the sensor measurement was collected. A location may be indicated by a coordinate set, which may include a measurement on each axis, e.g., a measurement on the X-axis and a measurement on the Y-axis. In further examples, table 650 may also include additional location measurements (e.g., a Z axis), time stamps of times when the data was collected, additional ambient environment measurements (e.g., those collected by other sensors, either passively or actively, and multiple measurements at a similar time and location), and/or analysis of ambient environment measurements (e.g., averages of the multiple measurements at a similar time and location). Alternative measurements are also possible. For example, robotic device 602 may determine that it is in Room 52 and store an identifier for room 52 in lieu of the X-axis and Y-axis measurements. In some examples, table 650 may be updated as robotic device 602 collects additional measurements. Robotic device 602 may store table 650 and periodically send reports of the ambient environment measurements to a remote device, where a user may analyze and verify the data.

As indicated in table 650, robotic device 602 may collect ambient environment measurements at the same point in time, but different locations (e.g., measurements in rows 652 and 654), as well as at the same location but different points in time (e.g., measurements in rows 652 and 662). In practice, the collected ambient environment measurements of table 650 may depend on the tasks that the robotic device is performing. For example, if the robotic device does not navigate to a certain location on a certain date to perform a task, then the measurements at that location may be absent from table 650.

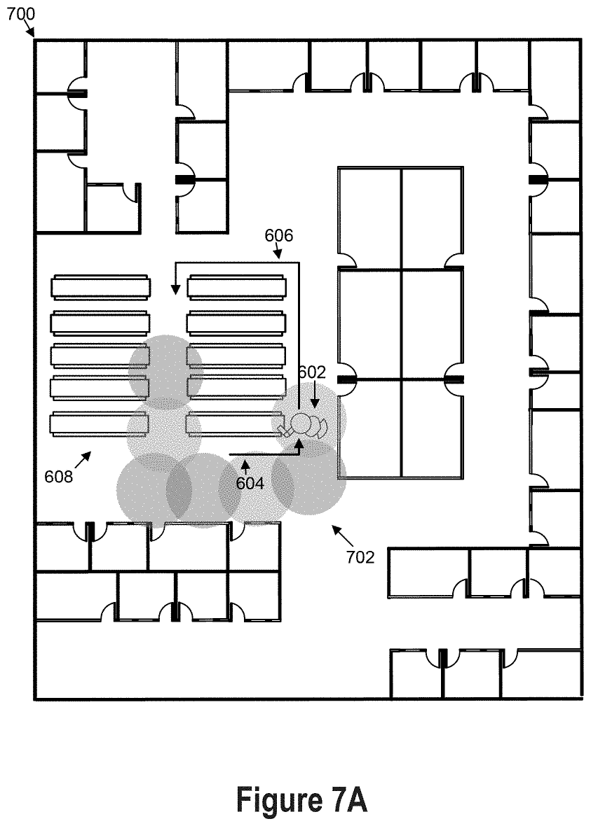

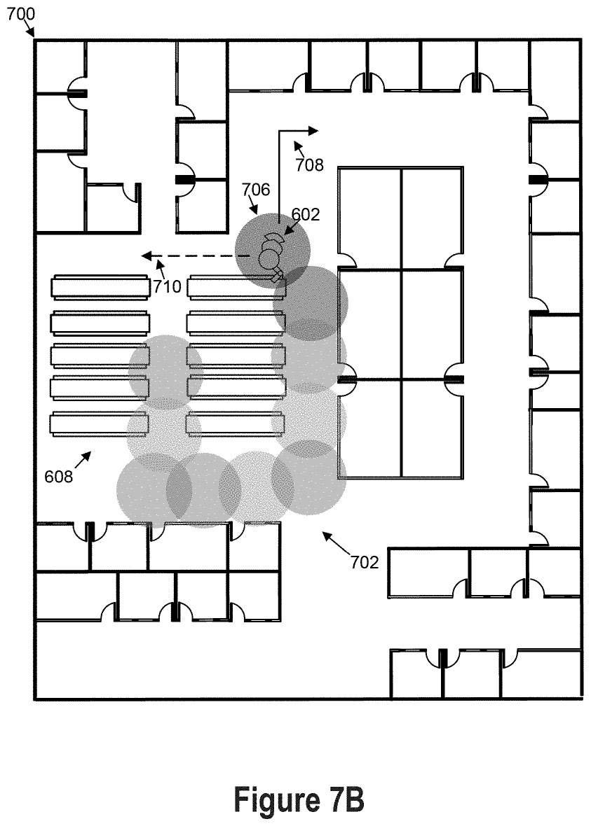

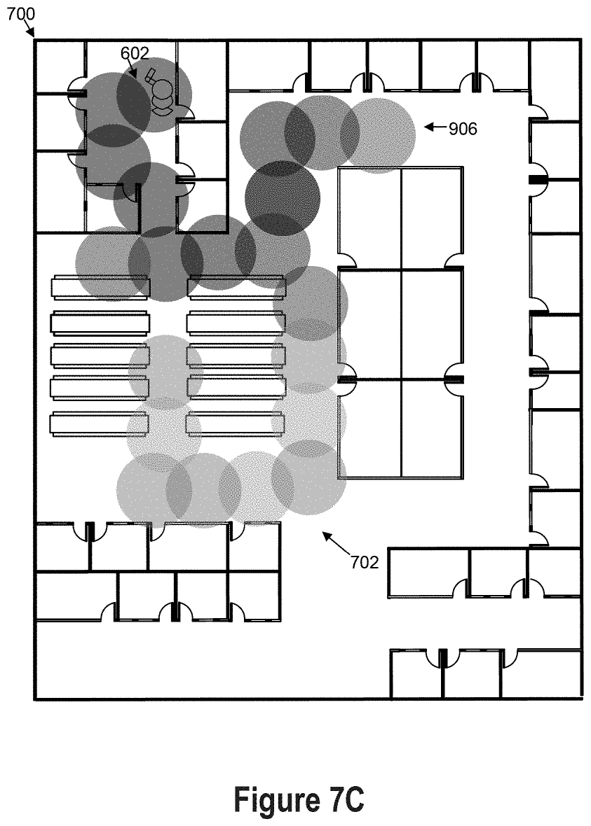

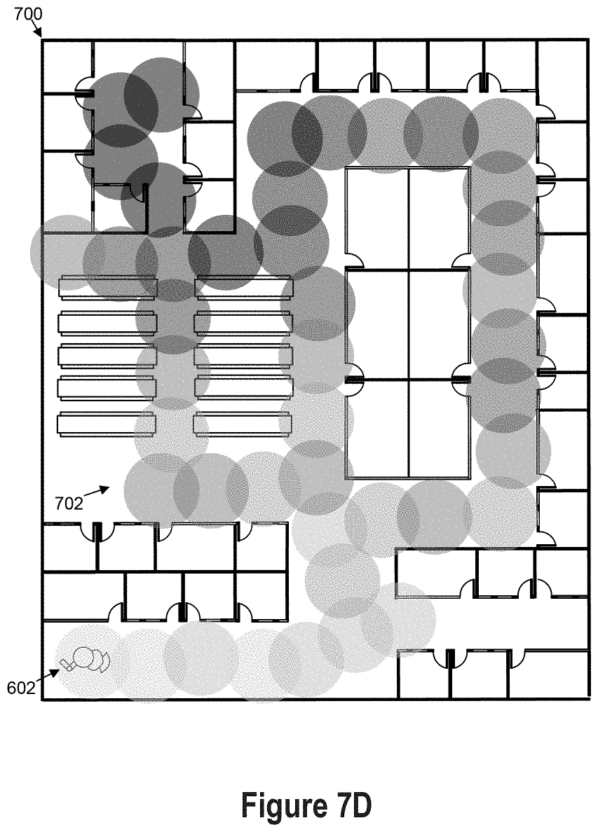

Referring back to FIG. 5, at block 504, method 500 includes determining the ambient environment state representation using the data collected by the at least one sensor on the robotic device. For example, the measurements in table 650 may be incorporated into an ambient environment state representation. FIG. 7A depicts ambient environment state representation 700, in accordance with example embodiments. Ambient environment state representation 700 may include heatmap 702 overlaid on a floorplan of a building in which robotic device 602 is operating.

Heatmap 702 may include representations of the measurements of table 650, as robotic device 602 moves along path 604 and path 606. In some examples, heatmap 702 may depict the ambient environment measurements using a color scheme that represents the relative high and/or low of the measurements. In further examples, heatmap 702 may include analysis of ambient environment measurements (e.g., averages of multiple samples collected around the same time) and/or may only include ambient environment measurements collected from a certain sensor and/or within a certain frame of time. Ambient environment measurements collected from further sensors and/or during another frame of time may perhaps be incorporated into additional heatmaps and/or additional ambient environment state representations.

Ambient environment representation 700 is an example representation of measurements in table 650, and many other examples are possible. For example, heatmap 702 may be overlaid on an image of the environment that the robotic device generates from a map used for navigation. This map of the environment may be based on sensor data collected from other sensors on the robotic device, such as one or more cameras and/or one or more LIDAR sensors. The ambient environment representation could also be three dimensional, mapping out X-axis, Y-axis, and Z-axis coordinates. Ambient environment representation 700 includes robot 602, path 604, and path 606 for demonstration purposes but in practice, the location of the robotic device and past/future paths of the robotic device may be excluded from ambient environment representation 700.

As robotic device 602 receives additional ambient environment measurements from the sensor, ambient environment representation 700 may be updated with the additional measurements. In some examples, ambient environment representation 700 may be generated periodically and may be sent to a remote device where a user may analyze the data. In further examples, ambient environment representation 700 may be generated once the robotic device detects one or more anomalous measurements (e.g., if a certain measurement is above a predetermined threshold). In still further examples, ambient environment representation 700 may be used to identify one or more anomalous measurements.

Referring back to FIG. 5, at block 506, method 500 includes identifying, based on the ambient environment state representation, one or more anomalous ambient environment measurements. As robotic device 602 navigates around environment 600, it may collect additional measurements of various ambient environment properties, and one or more of these additional measurements could be identified as anomalous. Anomalous ambient environment measurements may be measurements that indicate anomalies in the environment and/or measurements that are otherwise inconsistent with expected ambient environment measurements.

Ambient environment measurements may be identified as anomalous through various methods, including being based on previous measurements (e.g., based on the ambient environment representation), thresholds, and/or statistical tests. For example, referring back to FIG. 6B, the measurements of row 672 and row 674 may be flagged as being anomalous for being higher than measurements in rows 652, 654, 662, and 664. In other examples, the measurements of row 672 and row 674 may be designated as anomalous due to the measurements being above the normal CO2 and TVOC measurements of 700 ppm and 500 ppm, respectively. In further examples, the measurements of row 672 and 664 may be significantly higher than the measurements in row 652, row 654, row 662, and row 664 as indicated by a t-test or other statistical test.

Upon detecting one or more anomalous measurements, robotic device 602 may notify a remote device of the anomaly. For example, the robotic device could send an updated ambient environment representation 700, an updated table 650, or the one or more anomalous measurements. A user monitoring the remote device could then take action based on the notification. Alternatively or additionally, the user monitoring the remote device may send a notification back to the robotic device indicating whether the robotic device should take action and perhaps indicating what action the robotic device should take.

Referring back to FIG. 5, at block 508, method 500 includes causing, based on the one or more identified anomalous ambient environment measurements, the robotic device to actively monitor the environment such that robotic device navigation is based on the ambient environment state representation. FIG. 7B depicts example ambient environment representation 700, after detecting one or more anomalous measurements. Example ambient environment representation 700 includes heatmap 702, which includes anomalous measurement representation 706.

Before detecting the anomalous measurement represented by anomalous measurement representation 706, robotic device 602 may have intended to continue along path 606, perhaps in order to continue clearing tables 608. However, upon detecting the anomalous measurement, robotic device 602 may determine to navigate along path 708 to collect further ambient environment measurements.

In some examples, robotic device 602 may also use ambient environment state representation 700 to determine the direction and/or path in which to navigate. For example, robotic device 602 may be in a four-way intersection when it identifies the anomalous ambient environment measurement. Robotic device 602 may determine that, based on the ambient environment state representation, two paths in the four-way intersection yielded in less anomalous measurements. Thus, robotic device 602 may navigate in the direction of the third path in the four-way intersection.

In some examples, robotic device 602 may use past ambient state representations to determine the direction in which to navigate. For example, robotic device 602 may have collected ambient environment data for several days prior to discovering the anomalous measurement represented by anomalous measurement representation 706, and robotic device 602 may have aggregated the ambient environment data into one or more ambient environment representations. After having identified the anomalous measurement represented by anomalous measurement representation 706, robotic device 602 may analyze the past ambient environment state representations and determine that measurements along path 708 yielded lower magnitude measurements than measurements along path 710. In some examples, robotic device 602 may be navigating to determine a source of the anomalous measurements and measurements of a higher magnitude are more anomalous. Thus, robotic device 602 may navigate along path 710.

In further examples, robotic device 602 may also use the identified anomalous ambient environment measurements to determine the direction and/or path in which to navigate. For example, robotic device 602 may be navigating in order to discover a source of the anomalous measurements. Robotic device 602 may determine that the current direction in which it is traveling yielded in more anomalous measurements than a past direction. Thus, robotic device 602 may continue to navigate in that direction.

In some examples, robotic device 602 may also use additional measurements to determine the direction in which to navigate. For example, the sensor collecting the ambient environment measurements may be located on a moveable appendage of robotic device 602, and additional measurements may be collected through controlling the moveable appendage. Robotic device 602 may manipulate the moveable appendage to collect measurements in two or more directions. Depending on the application and what robotic device 602 is attempting to determine, robotic device 602 may determine the most or least anomalous measurement and move in the direction of that determined measurement. For example, if robotic device 602 is attempting to discover the source of the anomalous measurements, robotic device 602 may move in the direction of the most anomalous measurement.

In further examples, robotic device 602 may have an additional sensor to measure air flow and the navigation of the robotic device may be based on these measurements. For example, the air flow sensor may indicate that the direction of air flow is in the opposite direction as path 708. Robotic device 602 may thus determine that the source of the anomalous measurement may be in the direction of path 708 and navigate in the direction of path 708 in order to determine a source of the anomalous measurements.

In some examples, the robotic device may navigate towards path 708 in order to discover a source of the anomalous ambient environment measurements, but determine that the measurements in the direction of path 708 are decreasingly anomalous. Based on this determination, the robotic device may navigate to take path 710. FIG. 7C depicts ambient environment representation 700 in such a situation, in accordance with example embodiments. Robotic device 602 may have first navigated towards the location of the measurement indicated by ambient measurement representation 706. Robotic device 602 may have determined that these measurements were not increasingly anomalous and thus were not towards the source of the anomalous measurements. Based on these measurements, robotic device 602 may navigate in a different direction, for example towards the location of robot 602 as indicated in FIG. 7C.

Following navigation processes as outlined above, robotic device 602 may determine that the corner of the building in which it is located in FIG. 7C is the approximate location of the source of the anomalous measurements. In some examples, robotic device 602 may send an indication of this location, perhaps in conjunction with ambient environment representation 700 and/or table 650, to a remote device. A user monitoring the remote device then may take action based on this information.

In some examples, a robotic device may fine tune the determined location of the source of the anomalous measurements. For example, a sensor capable of detecting the ambient environment measurements may be located on a movable appendage of the robotic device. The robotic device may thus control the appendage, determine the approximate location of the sensor, and determine an ambient environment measurement. The appendage could be further controlled based on the magnitude of the ambient environment measurement.

In some examples, robotic device 602 may identify a possible source of the anomalous measurements. For example, robotic device 602 may have determined an anomalous air quality measurement and determined that the corner of the building in which it is located in FIG. 7C is the approximate location of the source of the anomalous measurements. Robotic device 602 may then determine images of the surroundings and identify objects in the images that could be a possible source of the anomalous measurement. For example, one of the images may contain a burning cigarette and another image may contain open windows. Robotic device 602 may determine that the air quality measurement is due to one or both of the two. Subsequently, robotic device 602 may send the images, identified objects, and/or location to a remote server such that a human may take action based on the data.

This identification process could also serve for verification that the robotic device identified the correct location of the anomalous measurements. For example, if robotic device 602 receives images of the location of the source and determines one or more images contain a possible source of the measurement, robotic device 602 may determine that this location is likely the source of the anomalous measurements. Alternatively, if none of the images contain a possible source of the measurement, robotic device 602 may determine that the location is unlikely the source of the anomalous measurements. Further action of the robotic device may be based on this determination.

In some examples, the robotic device 602 may then determine the downstream effects of the anomalous measurements. FIG. 7D depicts ambient environment representation 700 after determining the downstream effects of the anomalous measurements. Determining the downstream effects of the anomalous measurements may involve similar navigation processes as outlined above. For example, determining the downstream effects of the anomalous measurements may be based on ambient environment representations, additional ambient environment measurements, additional sensors, and/or other factors.

In some examples, the process of determining the downstream effects of the anomalous measurements may incorporate an additional air flow sensor. For example, robotic device 602 may incorporate both an air quality sensor and an air flow sensor. Upon detecting an anomalous air quality measurement, robotic device 602 may use the air flow sensor to determine a direction that the air is flowing from a location of the anomalous air quality measurement. Robotic device 602 may then navigate based on the air flow (e.g., in the direction of the air flow or opposite of the direction of the air flow). In further examples, after robotic device 602 has navigated based on the air flow, robotic device 602 may determine an air quality measurement. Further movement of robotic device 602 may be based on the air flow measurements and the air quality measurements. For example, if robotic device 602 moved in the opposite direction of the air flow but the air quality measurement is indicated to be decreasingly anomalous, then the robotic device 602 may change directions to move in the same direction of the air flow. Such principles could also be applied to other environment properties, e.g., using air flow sensors to determine an expected temperature gradient from one location to another.

In some examples, active monitoring may be temporary. For example, after obtaining a map of the downstream effects, and perhaps after sending the map of the downstream effects to a remote server, the robotic device could return to passively monitoring the environment. Put another way, the robotic device could return to navigating around the environment such that the navigation is not based on ambient environment measurements. In some situations, the robotic device may have been clearing tables before determining the one or more anomalous ambient environment measurements, and the robotic device could return to clearing tables after completing active monitoring of the environment, e.g., after obtaining a map of the downstream effects.

In some examples, after detecting the one or more anomalous ambient environment measurements (and perhaps after obtaining the map of the downstream effects), the robotic device could suggest steps to mitigate the anomalous ambient environment measurements. For example, if the robotic device determines that the air quality in the south wing of the building is worse than the rest of the building, the robotic device could send a report to a remote device listing potential steps that could be taken to mitigate the poor air quality (e.g., opening windows in the south wing, adding an air purifier, removing an identified source of contaminate).

In further examples, after detecting the one or more anomalous ambient environment measurements (and perhaps after obtaining the map of the downstream effects), the robotic device could take steps to mitigate the anomalous ambient environment measurements. For example, if the robotic device detects that the south wing of a building has an abnormally high temperature compared to the rest of the building, the robotic device could adjust the thermostat of the south wing to a lower temperature. Further, the robotic device could notify a user, perhaps through sending a signal to a remote device, that it took such an action.

In some examples, after detecting the one or more anomalous ambient environment measurements, the robotic device may record the anomalous measurements and after a predetermined amount of time, the robotic device may revisit the location to determine changes in the ambient environment property. The robotic device may record the anomalous measurements in table 650 and/or ambient environment representation 700 along with timestamps of the times at which the anomalous measurements were recorded and coordinate sets of the locations at which the anomalous measurements were recorded. After a predetermined amount of time following the timestamps, the robotic device may revisit the recorded location indicated by the coordinate sets and collect one or more additional measurements. The additional measurements may be compared to the anomalous measurements, threshold, or other previously mentioned method of analysis to determine whether the ambient environment property measurements are still anomalous, more anomalous, less anomalous, or no longer anomalous. For example, the robotic device may have detected an anomalous air quality measurement, e.g., a TVOC measurement of 550 ppm, at location X=20 and Y=20 at LOAM. At 3 PM, after a predetermined time period of five hours, the robotic device may return to location X=20 and Y=20, determine that the TVOC measurement is now 450 ppm, and that this measurement is not anomalous. In some examples, the additional measurements may be collected after a predetermined time period following the timestamp indicating the time at which the supposed cause of the anomaly was removed.

Further, the robotic device may record the anomalous measurements, perhaps with coordinate sets indicating locations and timestamps indicating times in table 650 and/or ambient environment representation 700. The robotic device may transmit these measurements in a query to a remote device for storage, perhaps to be later provided to an additional robotic device for further monitoring of the anomalous measurement location. For example, the additional robotic device may be operating in the same environment as the robotic device and may periodically retrieve information from the remote device, such as the anomalous measurements and the corresponding coordinate sets and timestamps. The additional robotic device may calculate, from a predetermined time period and the timestamps, times at which to re-evaluate the anomalous measurements. Additionally, the additional robotic device may navigate to the recorded locations indicated by the coordinate sets at the calculated times and determine whether the measurements are still anomalous, more anomalous, less anomalous, or no longer anomalous. The additional robotic device may then take action based on this determination.

### III. CONCLUSION

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims.

The above detailed description describes various features and functions of the disclosed systems, devices, and methods with reference to the accompanying figures. In the figures, similar symbols typically identify similar components, unless context dictates otherwise. The example embodiments described herein and in the figures are not meant to be limiting. Other embodiments can be utilized, and other changes can be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

A block that represents a processing of information may correspond to circuitry that can be configured to perform the specific logical functions of a herein-described method or technique. Alternatively or additionally, a block that represents a processing of information may correspond to a module, a segment, or a portion of program code (including related data). The program code may include one or more instructions executable by a processor for implementing specific logical functions or actions in the method or technique. The program code or related data may be stored on any type of computer readable medium such as a storage device including a disk or hard drive or other storage medium.

The computer readable medium may also include non-transitory computer readable media such as computer-readable media that stores data for short periods of time like register memory, processor cache, and random access memory (RAM). The computer readable media may also include non-transitory computer readable media that stores program code or data for longer periods of time, such as secondary or persistent long term storage, like read only memory (ROM), optical or magnetic disks, compact-disc read only memory (CD-ROM), for example. The computer readable media may also be any other volatile or non-volatile storage systems. A computer readable medium may be considered a computer readable storage medium, for example, or a tangible storage device.

Moreover, a block that represents one or more information transmissions may correspond to information transmissions between software or hardware modules in the same physical device. However, other information transmissions may be between software modules or hardware modules in different physical devices.

The particular arrangements shown in the figures should not be viewed as limiting. It should be understood that other embodiments can include more or less of each element shown in a given figure. Further, some of the illustrated elements can be combined or omitted. Yet further, an example embodiment can include elements that are not illustrated in the figures.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims.

What is claimed is:

1. A method comprising:
receiving data collected by at least one sensor on a robotic device, wherein the data is to be used for an ambient environment state representation, and wherein the data represents ambient environment measurements collected at locations of the at least one sensor when the robotic device is passively monitoring an environment such that robotic device navigation is not based on the ambient environment state representation;
determining the ambient environment state representation using the data collected by the at least one sensor on the robotic device;
identifying, based on the ambient environment state representation, one or more anomalous ambient environment measurements; and
causing, based on the one or more identified anomalous ambient environment measurements, the robotic device to actively monitor the environment such that robotic device navigation is based on the ambient environment state representation.

2. The method of claim 1, wherein the at least one sensor is a temperature sensor, a humidity sensor, a total volatile organic compound sensor, an air quality sensor, an air flow sensor, a particulate matter sensor, or a carbon dioxide sensor.

3. The method of claim 1, wherein the method further comprises:
receiving robotic device coordinates corresponding to robotic device locations where the ambient environment measurements are collected; and
determining, based on the robotic device coordinates, sensor coordinates corresponding to the locations of the at least one sensor, wherein determining the ambient environment state representation is further based on the sensor coordinates.

4. The method of claim 1, wherein the method further comprises:
receiving timestamps corresponding to when the data was collected, wherein determining the ambient environment state representation is further based on the timestamps.

5. The method of claim 1, wherein the method further comprises receiving a map of the environment, and wherein determining the ambient environment state representation using the data collected by the at least one sensor on the robotic device comprises:
determining a visual representation of the data, wherein the visual representation is indicative of relative magnitude of the ambient environment measurements; and
overlaying the visual representation onto the map of the environment.

6. The method of claim 1, wherein the method further comprises:
in response to identifying the one or more anomalous ambient environment measurements, transmitting, to a remote device, the ambient environment state representation.

7. The method of claim 1, wherein the method further comprises:
in response to identifying the one or more anomalous ambient environment measurements, transmitting, to a remote device, a notification indicating detection of the one or more anomalous ambient environment measurements.

8. The method of claim 1, wherein causing the robotic device to actively monitor the environment such that robotic device navigation is based on the ambient environment state representation comprises:
predicting, based on the ambient environment state representation, a direction towards a source of the one or more anomalous ambient environment measurements; and
navigating the robotic device based on the predicted direction.

9. The method of claim 1, wherein the method further comprises:
in response to identifying the one or more anomalous ambient environment measurements, determining a location of a source of the one or more anomalous ambient environment measurements; and
transmitting, to a remote device, the location of the source.

10. The method of claim 1, wherein the at least one sensor is located on an appendage of the robotic device, wherein causing the robotic device to actively monitor the environment such that robotic device navigation is based on the ambient environment state representation comprises:
predicting, based on the ambient environment state representation, a direction towards a source of the one or more anomalous ambient environment measurements; and
causing the robotic device to manipulate the appendage based on the predicted direction.

11. The method of claim 1, wherein the method further comprises:
after identifying the one or more anomalous ambient environment measurements, receiving, from another sensor on the robotic device, additional data indicative of a source of the one or more anomalous ambient environment measurements; and
transmitting, to a remote device, the additional data.

12. The method of claim 1, wherein, for each of the one or more anomalous ambient environment measurements, the method further comprises:
determining a coordinate set corresponding to an anomalous measurement location where the anomalous ambient environment measurement was collected;
receiving a timestamp corresponding to when the anomalous ambient environment measurement was collected; and
after a predetermined time period following the timestamp, causing the robotic device to navigate to the anomalous measurement location indicated by the coordinate set.

13. The method of claim 1, wherein, for each of the one or more anomalous ambient environment measurements, the method further comprises:
determining a coordinate set corresponding to an anomalous measurement location where the anomalous ambient environment measurement was collected;
receiving a timestamp corresponding to when the anomalous ambient environment measurements was collected;
transmitting, to a remote device, a query including at least the coordinate set and the timestamp, wherein the coordinate set and timestamp are provided to an additional robotic device to further monitor the anomalous measurement location after a predetermined time period following the timestamp.

14. A robotic device comprising:
at least one sensor; and
a control system configured to:
receive data collected by the at least one sensor on the robotic device, wherein the data is to be used for an ambient environment state representation, and wherein the data represents ambient environment measurements collected at locations of the at least one sensor when the robotic device is passively monitoring an environment such that robotic device navigation is not based on the ambient environment state representation;
determine the ambient environment state representation using the data collected by the at least one sensor on the robotic device;
identify, based on the ambient environment state representation, one or more anomalous ambient environment measurements; and
cause, based on the one or more identified anomalous ambient environment measurements, the robotic device to actively monitor the environment such that robotic device navigation is based on the ambient environment state representation.

15. The robotic device of claim 14, wherein the robotic device navigation, when the robotic device is passively monitoring the environment, is based on a predetermined path.

16. The robotic device of claim 14, wherein the robotic device further comprises an additional sensor, wherein the robotic device navigation, when the robotic device is passively monitoring the environment, is based on additional measurements from the additional sensor.

17. The robotic device of claim 14, wherein the at least one sensor is an air quality sensor, wherein the robotic device further comprises an air flow sensor, wherein the one or more anomalous measurements are one or more anomalous air quality measurements, and wherein the control system is further configured to:
in response to identifying the one or more anomalous air quality measurements, determine a location of a source of the one or more anomalous air quality measurements;
determine, based on air flow measurements from the air flow sensor, an air flow direction while the robotic device is in the location of the source of the one or more anomalous air quality measurements; and
cause the robotic device to navigate based on the determined air flow to determine a downstream effect of the one or more anomalous air quality measurements.

18. The robotic device of claim 14, wherein the robotic device further comprises a camera, wherein the control system is further configured to:
in response to identifying the one or more anomalous ambient environment measurements, determine a location of a source of the one or more anomalous ambient environment measurements;
receive one or more images from the camera, wherein the one or more images are indicative of a source of the one or more anomalous ambient environment measurements; and
verify, based on the images, the location of the source of the one or more anomalous environment measurements.

19. The robotic device of claim 14, wherein the at least one sensor is at least one interchangeable sensor, and wherein the control system is further configured to:
in response to identifying the one or more anomalous ambient environment measurements, cause the robotic device to remove the at least one interchangeable sensor; and
cause the robotic device to replace the at least one interchangeable sensor with an additional sensor.

20. A non-transitory computer readable medium comprising program instructions executable by at least one processor to cause the at least one processor to perform functions comprising:
receiving data collected by at least one sensor on a robotic device, wherein the data is to be used for an ambient environment state representation, and wherein the data represents ambient environment measurements collected at locations of the at least one sensor when the robotic device is passively monitoring an environment such that robotic device navigation is not based on the ambient environment state representation;
determining the ambient environment state representation using the data collected by the at least one sensor on the robotic device;
identifying, based on the ambient environment state representation, one or more anomalous ambient environment measurements; and
causing, based on the one or more identified anomalous ambient environment measurements, the robotic device to actively monitor the environment such that robotic device navigation is based on the ambient environment state representation.
